(19) United States
(12) Patent Application Publication
(10) Pub. No.: US 2012/0016347 A1
(43) Pub. Date: Jan. 19, 2012

(54) Seroma Control for Surgical Patients

(76) Inventors: John I. Shipp, Atlantic Beach, FL (US); B. Todd Heniford, Charlotte, NC (US)

(21) Appl. No.: 12/835,132

(22) Filed: Jul. 13, 2010

(51) Int. Cl. A61M 27/00; A61K 38/48; A61K 33/12

(52) U.S. Cl. 604/541; 424/683; 424/94.64

(57) ABSTRACT

Apparatus and methods for significant reduction of seroma formation, seroma related complications and time until drain removal following surgery are disclosed. Talc, with and without thrombin, bleomycin, erythromycin, tetracycline, doxycycline, polidocanol alone or in combination or combined with talc and/or thrombin for use in operatives wound and lymph node dissections to reduce seromas are disclosed.

### FIELD OF THE INVENTION

The present invention relates generally to control of surgical drainage and seroma prevention in surgical and trauma wounds and, in particular, to applying talc in the wounds to induce a localized inflammatory reaction to reduce the fluids that may accumulate.

### DESCRIPTION OF THE RELATED ART

It will be appreciated by those skilled in the art that talc, doxycycline, tetracycline, erythromycin, bleomycin, and polidocanol can be used to treat retained fluid and effusions in the chest cavity and pleural space. With the fluid drained by various methods, the administration of all of these agents results in a controlled, local inflammatory response that seals the lung to the chest wall and prevents fluid re-accumulation.

Surgical procedures that require a significant soft tissue dissection and/or lymph node resection result in loss of fluid from the cut surface of those tissues or weeping of lymphatic fluid. The collection of this tissue or lymphatic fluids under a patient's skin or within the surgical wound, otherwise known as a seroma, can lead to wound breakdown, infection, discomfort, scarring, or other chronic wound issues and the need for repeated drainage procedures or surgical operations. Currently, near the completion of an operation, surgeons place drainage tubes in the operative tissues to collect this fluid in an attempt to prevent a seroma from occurring. Over the past three decades better wound preparation, and improved tissue coagulation instruments and surgical techniques have done little to reduce seroma development and its subsequent complications.

Pleural effusions differ significantly from seromas both by location, pathophysiology, and indications for treatment. The pleural space is a closed serous sac. The pleura consists of two parts, the parietal pleura, which is applied to the thoracic wall, and the visceral pleura, which covers the surface lung. Each of these pleural surfaces is lined by a mesothelial layer that constantly secretes fluid. This fluid acts as a lubricant that allows the lung to slide or move along the inner chest cavity during the process of breathing. Finding fluid in this space is normal, and, in fact, it is estimated that more than 5000 ml of fluid transgresses this space every day in adults. Pleural effusions, an abnormal collection of fluid in the pleural space, typically results from cancer, either primary or metastatic. The fluid that normally exists in the pleural space is not collected by the pleura, has a dramatic increase in protein content due to increased capillary permeability, and impacts the patient's ability to breath by compressing the lung.

Seroma fluid originates primarily from weeping fluid due to direct tissue trauma inflicted at the time of operation and from surgically disrupted lymphatic channels. The seroma fluid collects in the area of the operative field and is not typically found “free” in these areas. Unlike the chest cavity, the space where the seroma fluid collects does not exist until an operation opens it. Seromas are not directly caused by cancer, but may be secondary to the surgical treatment of cancer.

The wounds from surgery and trauma provide the tissue injury that results in the accumulation of wound fluid and seroma formation. It is also well know by those skilled in the art that seromas lead to surgical wound complications in operations such as hernia repairs, breast cancer procedures, lymph node resections, hip replacements, free tissue flaps, panniculectomies, tummy tucks, soft tissue and muscles flaps and transfers, and others. If these fluid collections are not controlled, high levels of morbidity can result. Often, surgeons place drainage tubes in the patients' wounds to remove the fluid. However, the drain tubes frequently fail, become infected themselves, or are required to be in place for weeks post-operatively resulting in discomfort, scarring, and other problems along with a reduction in patient quality of life.

Seroma formation following complex procedures such as ventral hernia repair, orthopedic, plastic surgery and cancer operations are serious, painful, expensive to treat, and can lead to additional serious complications. The very minor to major infection rates following these procedures can exceed 30 percent. An additional problem of postoperative care is a huge cost to the health care system, the treatment of which can exceed the cost of the original surgery.

What is needed then is a method of lowering the incidence of seromas following surgery by providing an effective agent at the site of the wound at the time of the surgery or trauma that is non-toxic and results in rapid tissue attachment which eliminates tissue/lymphatic weeping in the space in which the seroma forms. The method of reduction in the rate of seroma formation will most likely allow earlier removal of drains, therefore, reduce the complications associated with the drains themselves and improve patient quality of life.

### Quantitative Complication Reduction

Those skilled in the art are aware that sterile talc, doxycycline, tetracycline, bleomycin, erythromycin and polidocanol are effective agents to treat pleural effusions but do not respond to simple drainage. Talc has been utilized for many years. Talc as a pure chemical compound is defined as hydrous magnesium silicate, Mg₃Si₄O₁₀(OH)₂. A variety of elements such as nickel and iron may be included in the talc particle lattice, but are so bound within the particle that they are not free to exert any biological action. (Gross and Harley, 1973). Talc can be tabular, granular, fibrous, or platy, but it is usually crystalline, flexible, and soft. As described by Colt and many others, talc is effective in 80%-90% of cases with a safe adverse-event profile. (Colt—The Lancet—Oncology; Vol 9, Page 912; October, 2009). Of the more than 30 randomized, prospective trials, most favor talc to treat recurrent pleural fluid collections. Indeed, the Cochrane review ranks it as the pleurodesis agent of choice [Shaw P, Agarwal R. Pleurodesis for malignant pleural effusions (Cochrane Review). Cochrane Database Syst Rev 2004; CD002916.]. It can be effective when administered by either poudrage or slurry. It can be used as large particle, small particle, or a combination of both. Small particle talc has been associated with greater rate of complications when used in the pleural space of the chest and is related to adult respiratory syndrome due to the direct absorption.

Erythromycin is primarily used as an oral antibiotic to treat gram-positive bacteria. It has been utilized as a pleural sclerosing agent. When placed in the pleural space it can result in adherence of the lung to the chest wall. The use of erythromycin may have the advantage of a reduction in inflammation as described by Miller (J of Surg Education; Vol 64, No.1, January 2007), but the extent of fibrosis remains high, which results in the fibrosis and the desired effect of collapse of the pleural space. Its first use in recurrent pleural fluid collections was in 1935. Its use is uncommon in clinical practice and there is limited human data. Doxycycline and tetracycline are primarily used as antibiotics taken orally. The application of these agents for retained or recurrent pleural fluid collections has been described as safe for many years. There are several descriptions of their success. Patz (Chest, 1998) demonstrated a 79% success rate in completely or partially controlling pleural effusions with doxyclycline. In a head-to-head study published in the Medical Science Monitor in 2004 (Kuzdal, et.al. Management of Recurrent Malignant Pleural Effusions with Chemical Pleurodesis), doxycycline did not perform as well as talc, which had significantly higher rates of response both short and long term. Miller described in an animal model that Doxycycline may cause more inflammation than other agents. (J of Surg Education; Vol 64, No. 1, January 2007).

Bleomycin, a chemotherapy medication, appears to have few side effects, but is somewhat less effective and more expensive when compared to talc for pluerodesis. This was documented in a retrospective review by Kilic (Surgery Today 2004) when their group that used talc allowed for earlier drain removal, fastest lung re-expansion, and greater overall success than bleomycin. Patz (Chest, 1998) demonstrated a 72% success rate in completely or partially controlling pleural effusions with bleomycin.

Polidocanol has typically been used as a sclerosing agent for extremity or esophageal varices. It is relatively less well studied for pluerodesis than the other agents. Cetin (Surgery Today 2003) did report that it compared well to tetracycline in animal models in the control of pleural fluid accumulation. No prospective studies in humans are available.

Combination of talc with other agents, such as doxycycline, has demonstrated good results in animal studies. Dikensoy's study of these combined showed statistically higher adhesion scores than either of these agents alone. (Chest, 2005). The possibility of a combination of other agents with talc, such as thrombin (a hemostatic agent), also exist.

The formation of seromas in post-surgical wounds such as hernia repair, lymph node resection, tummy tuck, panniculectomy, free or attached flaps, tissue transfers, and other major operations with a large dissection is ubiquitous unless drains are placed at the time of surgery. Despite drain placement, seromas still form, and the subsequent complication rates and reduction in patient quality of life can be high. Wound infection/cellulitis can exceed 30%, and office, operative or radiographic intervention can be required in 20% of patients.

The pathophysiology and location of pleural effusions and seromas are dramatically different. As well, pleurodesis as a preemptive treatment for an anticipated pleural effusion does not occur. In this patent, materials and methods for preemptive therapy for the prevention of seromas is described. These materials and methods are shown to be very effective in the treatment of this troublesome problem.

An anti-seroma agent would ideally be placed into the wound in question at the time of the original surgery. The goal would be to induce a very localized inflammatory reaction that would cause the weeping, cut surfaces of the wound to stick to surrounding tissues, thereby sealing them and the lymphatic vessels, while at the same time eliminating the space in which the fluid accumulates. By doing so, the wounds can heal and the surgical drains can be removed much earlier than previously, subsequently decreasing wound and drain-related complications.

### SUMMARY OF THE INVENTION

The present invention is directed to a method for controlling seromas in non-pleural spaces in a mammal that includes performing a surgical procedure in a non-pleural space, the surgical procedure creating at least one subcutaneous tissue surface, applying talc to the at least one subcutaneous tissue surface to induce an inflammatory response on the at least one subcutaneous tissue surface, placing a drain in the non-pleural space, and closing the non-pleural space.

Additional features and advantages of the invention will be set forth in the detailed description which follows, and in part will be readily apparent to those skilled in the art from that description or recognized by practicing the invention as described herein, including the detailed description which follows and the claims.

It is to be understood that both the foregoing general description and the following detailed description of the present embodiments of the invention are intended to provide an overview or framework for understanding the nature and character of the invention as it is claimed.

### DETAILED DESCRIPTION OF THE PREFERRED EMBODIMENTS

We performed a study evaluating a novel intra-operative technique of applying talc to the subcutaneous flaps created during panniculetomies, tummy tucks and hernia repair to prevent seroma formation. Following the operative dissection and otherwise completion of the operation except for wound closure, talc is sprayed into the wound in volumes from 4 grams to 8 grams. A large particle or mixed large and small particle talc were sprayed on all surfaces of the wound. This was performed with and without applying the hemostatic agent thrombin.

The data were collected prospectively identifying all patients undergoing these operations. Patients were divided into two groups based on receiving talc therapy. The PRE group did not receive talc therapy, and the POST group did receive talc therapy in the subcutaneous dissection. Patient demographics, peri-operative data, and outcomes were analyzed using standard statistical methods. The PRE group consisted of 108 patients and the POST group consisted of 61 patients. Patient demographic and peri-operative data, including patient age, race proportions, comorbidities, the type and extent of subcutaneous procedures, size of hernias, operative time, and others factors were similar between groups. Complication rates for PRE/ POST were: cellulites/oral antibiotics 39%/15%, intravenous antibiotics 10%/3%, operative/radiologic intervention for wound infection 22%/13%, seroma intervention 20%/4%, wound breakdown 11%/2%, and hernia recurrence 10%/0%. Of these, the incidence of cellulitis, antibiotic use, seroma intervention, wound complications, and hernia recurrence were significantly decreased in the POST group (p<0.05 in all groups). Mean drain duration was 28.4 days for PRE and 15.3 days for POST (p=0.0009).

The addition of talc or talc and thrombin made a dramatic difference in patient outcomes. It reduced the risk of infection, the need for antibiotics, significant wound issues, and the need for instrumentation or operation on seromas. As well, the patients' drains were removed remarkably early after the procedure. The wounds were closed with 4 grams of talc early in the study. Eight grams of talc were used later. The data from these time periods indicates that there may be a dose dependant outcome. The last 10 patients received 8 grams in their wounds and each had their drains removed by 12 days after the surgery. The addition of thrombin may be additive, given its hemostatic properties, but its exact contribution is not known.

### Example

### Wound Treatment

Surgical treatment of very large ventral hernias with concomitant panniculectomies or massive subcutaneous dissections were performed.

The typical operation included a wide skin and subcutaneous incision with significant subcutaneous dissection with and without skin resection. The ventral abdomen was entered, and the intestinal contents of the hernia were reduced back into the abdomen. The hernia was repaired with mesh, either placed under the muscular abdominal wall or on top of it. The subcutaneous tissues were irrigated and either closed or talc was sprayed or instilled in the wound with or without a coagulant (thrombin) prior to closure.

Closure of the abdomen was performed in the same manner in all patients regarding the sutures used and drains placed. Two flat Jackson-Pratt type drains were placed through the skin and into the subcutaneous space. They were left in place until less than 30 cc of fluid was drained over 24 hours. When either or both of the drains collected less than 30 cc of fluid, the drains were removed.

There were 169 patients were that divided into two groups based on whether or not they received talc therapy.

Following the prospective collection of data (patient demographics, peri-operative findings, and outcomes), the data were statistically analyzed.

### Patient Outcomes

The group that did not receive talc included 108 patients while the talc group included 61 patients. Patient demographic and peri-operative data, including patient age, race proportions, comorbidities, the type and extent of subcutaneous procedures, size of hernias, operative time, and others factors were similar between groups.

Complication rates for No Talc/Talc groups were: cellulites requiring oral antibiotics: 39%/15%; intravenous antibiotics: 10%/3%; operative/radiologic intervention for wound infection: 22%/13%; seroma intervention: 20%/4%; wound breakdown: 11%/2%; and hernia recurrence: 10%/0%. Of these, incidence of cellulitis, antibiotic use, seroma intervention, wound complications, and hernia recurrence were significantly decreased in the group of patients receiving talc (p<0.05 in all groups).

The subcutaneous drains that were placed at the time of surgery were removed much sooner in the patients that had received talc therapy. Mean drain duration was 28.4 days for patients that did not receive talc. It was 15.3 days for patients receiving talc in their subcutaneous space prior to wound closure (p=0.0009).

### Method of Use—Additional Sclerosing Agents

Following dissection and required operative procedures for paniculectomy, tummy tuck, ventral hernia repair, free or attached flap procedures, or other procedures that require broad dissection in the subcutaneous plane or other body areas, bleomycin, erythromycin, tetracycline, doxycycline, polidocanol alone or in combination or combined with talc and/or thrombin, is to be placed in the wound via spray, directly, or as a slurry. The standard drain placement and closure follow. The incidence of seroma formation and the need for prolonged drains in the wound should be minimized.

### Method of Use—Talc Used in Lymph Node Resection

Following standard resection of axillary, groin, peri-iliac, neck lymph nodes, or other lymph node barring areas, 2-4 grams of talc, with or without thrombin, is to be placed in the wound via spray, directly, or as a slurry. The standard drain placement and closure follow. The incidence of seroma formation and the need for prolonged drains in the wound was minimized.

### Method of Use—Additional Sclerosing Agents Used in Lymph Node Resection

Following standard resection of axillary, groin, peri-iliac, neck lymph nodes, or other lymph node barring areas, bleomycin, erythromycin, tetracycline, doxycycline, polidocanol alone or in combination or combined with talc and/or thrombin, is to be placed in the wound via spray, directly, or as a slurry. The standard drain placement and closure follow. The incidence of seroma formation and the need for prolonged drains in the wound was minimized.

I claim:

1. A method for controlling seromas in non-pleural spaces in a mammal comprising:
performing a surgical procedure in a non-pleural space, the surgical procedure creating at least one subcutaneous tissue surface;
applying talc to the at least one subcutaneous tissue surface to induce an inflammatory response on the at least one subcutaneous tissue surface; and
closing the non-pleural space.

2. The method according to claim 1, further comprising the step of applying a hemostatic agent on the at least one subcutaneous tissue surface.

3. The method according to claim 2, wherein the hemostatic agent is thrombin.

4. The method according to claim 1, where in the talc is sprayed on the at least one subcutaneous tissue surface.

5. The method according to claim 1, wherein the talc is applied to the at least one subcutaneous tissue surface in a slurry.

6. The method according to claim 1, where in the talc is applied in an amount of between about 4 grams and about 8 grams.

7. The method according to claim 1, further comprising the step of applying a sclerosing agent to the at least one subcutaneous tissue surface.

8. The method according to claim 2, wherein the sclerosing agent is selected from the group of sclerosing agents comprising bleomycin, erythromycin, tetracycline, doxycycline, and polidocanol.

9. The method according to claim 1, further comprising the step of placing a drain in the non-pleural space prior to closing the non-pleural space.
